(19) United States
(12) Patent Application Publication
(10) Pub. No.: US 2026/0253211 A1
(43) Pub. Date: Aug. 27, 2026

(54) MEDICAL IMAGE PROCESSING APPARATUS AND METHOD

(71) Applicant: CANON MEDICAL SYSTEMS CORPORATION, Otawara-shi (JP)

(72) Inventors: Keith Antony GOATMAN, Edinburgh (GB); Salman MOHAMMADI, Edinburgh (GB); Shadia MIKHAEL, Edinburgh (GB)

(73) Assignee: CANON MEDICAL SYSTEMS CORPORATION, Otawara-shi (JP)

(21) Appl. No.: 19/065,242

(22) Filed: Feb. 27, 2025

(51) Int. Cl. G06T 7/00; G06T 7/11; G16H 10/60; G16H 50/20

(52) U.S. Cl. CPC G06T 7/0012; G06T 7/11; G16H 10/60; G16H 50/20; G06T 2207/10081; G06T 2207/30008

(57) ABSTRACT

A medical imaging apparatus includes processing circuitry to receive a medical imaging data set comprising intensity values as a function of position; obtain a segmentation of a bone region represented in the medical imaging data set; process the medical imaging data set to identify a plurality of sub-regions within the bone region for which intensity values are within a threshold intensity range with respect to a calibration intensity value; and determine concentration and/or distribution of the sub-regions within the bone region.

### Field

Embodiments described herein relate generally to a method of processing medical image data, for example to a method of processing medical image data in order to analyse bone quality for assessing osteoporosis and fracture risk.

### BACKGROUND

Some existing approaches to estimate bone mineral density (BMD) use attenuation values (for example, measured in Hounsfield Units (HU)) to estimate the BMD. The mean value in HU within a region or segmentation of a specific vertebra may be used. A problem with this approach is that it is scanner and/or protocol specific. While it may work acceptably for a single protocol on a single scanner, scanners are usually not calibrated for high-density material such as bone, and HU values vary considerably between scanners and protocols, for example, for different values of kilovoltage peak (kVp) applied to an x-ray tube.

Osteoporosis, or ‘porous bone disease’ is a pathological reduction in bone density and strength either because of low production of new bone tissue or high destruction of old bone tissue, or both. Osteoporosis is a serious and widespread health problem. One in three women and one in five men over the age of fifty will suffer a fracture secondary to osteoporosis. Furthermore, 19% of women with osteoporosis after a recent vertebral fracture will sustain a new vertebral fracture within the next twelve months.

Following a hip fracture, men and women are more likely to die sooner, particularly if they are older at the time of the fracture. This increased mortality risk continues beyond a decade after the fracture. Although the prevalence of osteoporosis in women is higher than in men, about 30-40% of osteoporotic fractures occur in men, who also present with a higher mortality risk following a fracture at any site. In women over 45, fractures due to osteoporosis result in more in-patient hospital days than diabetes, heart attack and breast cancer.

BMD is an important clinical factor and one of multiple contributors to the risk of fracture. Trabecular bone score (TBS) or T-score may be computed from Dual-energy X-ray absorption (DEXA) images and provides a measure of the structural quality of bone tissue. DEXA is a low dose, dual-energy X-ray projection imaging technology which is commonly used to make routine quantitative measurements of BMD.

Computer tomography (CT) imaging delivers a higher radiation dose than DEXA and so is not expected to replace DEXA in routine testing for osteoporosis. CT imaging has advantages for assessing trabecular bone, such as the ability to exclude cortical bone, disambiguation of osteophytes and a true 3D distribution that allows detection of smaller holes or cavities in the trabecular structure than DEXA.

It is preferred to detect bone degeneration and intervene early. Radiologists fail to consistently review/report on the spine and 34-55% of spinal fractures are not reported in scans. Unfortunately, standard single-energy CT images do not reliably represent bone mineral density (BMD).

CT scanners are usually calibrated such that water at standard temperature and pressure has a value of zero Hounsfield Units (HU) and air has a value of-1000HU. Outside of these two calibration points the measured HU value depends on the X-ray beam energy spectrum and X-ray detector characteristics, and the differences can be large for higher attenuation material such as bone. In addition, effects such as variable beam hardening in differently shaped subjects mean that the measured HU value for a particular voxel of a CT image is also dependent on the subject's body composition elsewhere. The 0 HU value is particularly relevant, as pathologies that cause degeneration of bone tissue, such as osteoporosis, can result in lower density areas which may appear close to 0 HU and below in an image obtained from a CT scanner.

It is known, in quantitative CT (QCT) techniques, to place a calibration phantom in every scan. However this it is not useful for scans that are not directed specifically at bone density assessment.

### BRIEF DESCRIPTION OF THE DRAWINGS

Embodiments are now described, by way of non-limiting example, and are illustrated in the following figures, in which:

FIG. 1 is a schematic drawing of an apparatus according to an embodiment;

FIG. 2 is a flow chart illustrating operation of the apparatus of FIG. 1 according to an embodiment;

FIG. 3 (a), 3 (b) and 3 (c) show three medical images with low attenuation voxels labelled;

FIG. 4 is a plot of T-value versus percentage of voxels with low attenuation; and

FIG. 5 is a plot of bone mineral density versus percentage of voxels with low attenuation.

### DETAILED DESCRIPTION

In certain embodiments there is provided a medical imaging apparatus comprising processing circuitry configured to:
receive a medical imaging data set comprising intensity values as a function of position;
obtain a segmentation of a bone region represented in the medical imaging data set;
process the medical imaging data set to identify a plurality of sub-regions within the bone region for which intensity values are within a threshold intensity range with respect to a calibration intensity value; and
determine concentration and/or distribution of the sub-regions within the bone region.

In certain embodiments there is provided a medical imaging processing method comprising:
receiving a medical imaging data set comprising intensity values as a function of position;
obtaining a segmentation of a bone region represented in the medical imaging data set;
processing the medical imaging data set to identify a plurality of sub-regions within the bone region for which intensity values are within a threshold intensity range with respect to a calibration intensity value; and
determining concentration and/or distribution of the sub-regions within the bone region.

Chest and abdomen CT scans performed for a variety of purposes usually include images of the spine. CT scans where the spine is not the primary anatomy under investigation, such as chest and abdomen scans, present an opportunity for providing information about bone degeneration in the spine.

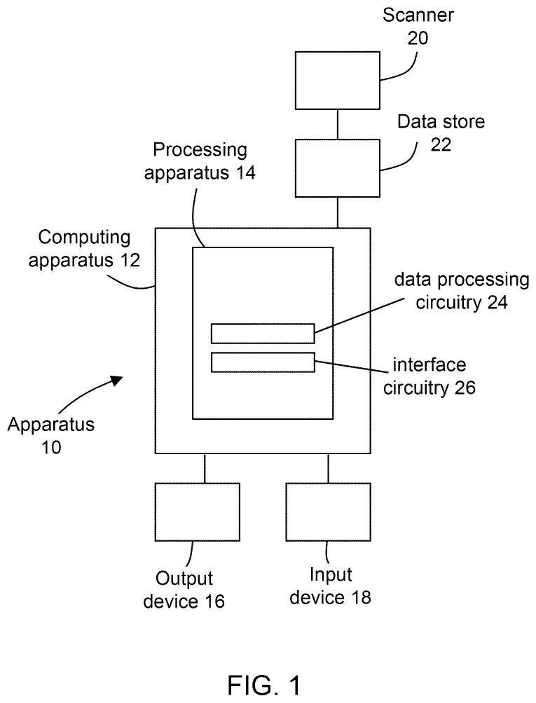

A data processing apparatus 10 according to an embodiment is illustrated schematically in FIG. 1. In the present embodiment, the data processing apparatus 10 is configured to process medical image data, such as imaging data from a CT scan. In other embodiments, the data processing apparatus 10 may be configured to process any other appropriate image data.

The data processing apparatus 10 comprises a computing apparatus 12, which in this example is a personal computer (PC) or workstation. The computing apparatus 12 is connected to one or more output devices 16, such as a screen or other display device, and one or more input devices 18, such as a computer keyboard and mouse.

The computing apparatus 12 is configured to obtain data sets from a data store 22. At least some of the data obtained from the data store comprises medical imaging data, for instance data obtained using a scanner 20. The medical image data may comprise two-, three- or four-dimensional data in any imaging modality. For example, the scanner 20 may comprise a CT (computed tomography) scanner, micro-CT scanner, cone-beam CT scanner, photon counting CT scanner or any suitable tomographic 3D X-ray scanner.

The computing apparatus 12 may receive data from one or more further data stores (not shown) instead of or in addition to data store 22. For example, the computing apparatus 12 may receive medical image data from one or more remote data stores (not shown) which may form part of a Picture Archiving and Communication System (PACS) or other information system.

Computing apparatus 12 provides a processing resource for automatically or semi-automatically processing the data. Computing apparatus 12 comprises a processing apparatus 14. The processing apparatus 14 comprises data processing circuitry 24, and interface circuitry 26 configured to obtain user or other inputs and/or to output results of the data processing. The data processing circuitry is configured to perform processing tasks, for example one or more of image segmentation, classification, image captioning, display and/or automated reporting or any other suitable task.

In the present embodiment, the circuitries 24 and 26 are each implemented in computing apparatus 12 by means of a computer program having computer-readable instructions that are executable to perform the method of the embodiment. However, in other embodiments, the various circuitries may be implemented as one or more ASICs (application specific integrated circuits) or FPGAs (field programmable gate arrays).

The computing apparatus 12 also includes a hard drive and other components of a PC including RAM, ROM, a data bus, an operating system including various device drivers, and hardware devices including a graphics card. Such components are not shown in FIG. 1 for clarity.

The data processing apparatus 10 of FIG. 1 is configured to perform methods as illustrated and/or described in the following.

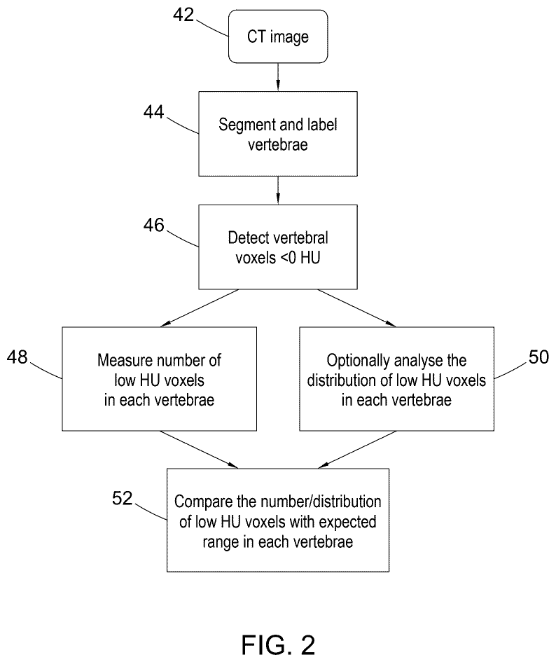

FIG. 2 illustrates a method of processing medical image data according to an embodiment, in which the medical image data is processed in order to assess bone quality.

At stage 42, medical image data is acquired and provided to the processing circuitry.

In the present embodiment, the medical image data includes data representing bone tissue. The images may be acquired using one of a number of appropriate imaging technologies and in this embodiment, the images comprise computer tomography (CT) images. The image data may be acquired using the scanner 20 or any other suitable scanner. The image data may be directly provided directly to the processing apparatus from the scanner 20 or may be received from the data store 22 or other source.

Any suitable form of medical image data may be used, for example the medical image data may comprise intensity values and/or attenuation values as a function of position. The medical image data may include semantic data associated with features represented in the image data in some embodiments.

The image data used in the example process describe in relation to FIG. 2 comprises three-dimensional image data comprising voxels. In other embodiments, the image data may be two-dimensional data comprising pixels. The two-dimensional image data may, for instance, comprise a two-dimensional slice from a tomographically acquired three-dimensional CT image.

At stage 44 a segmentation process is performed on the medical image data to segment regions of bone in the image data. The segmentation of the image data may comprise selecting pixels/voxels that are associated with one or more distinct pieces of bone tissue. Any suitable segmentation process can be used, for example in accordance with known techniques. The segmentation process may for example include labelling different bones and/or defining the boundaries of particular bones. Any suitable segmentation process may be used, for example any suitable rigid or non-rigid segmentation or any other segmentation process using any suitable segmentation technique.

In other embodiments, the image data may be pre-processed in order to segment the bone tissue and the segmentation, as well as the image data, may be provided to the processing apparatus 14, for example from the data store 22.

In the example described in relation to FIG. 2, the bone regions comprise one or more vertebrae included in some or all of the spinal column. The segmentation in this example includes labels for the segmented vertebral bones, chosen from a labelling scheme for vertebral bones.

At stage 46 the processing circuitry identifies a plurality of sub-regions within the bone regions for which intensity values are within a threshold intensity range about a calibration intensity value. The threshold intensity range may, for example, be between 0 HU and 100 HU in one example, but any other suitable intensity range may be chosen in other embodiments, for example within 1 HU or 10 HU or 50 HU or 100 HU of the calibration value in various embodiments.

In the present example each sub-region is a voxel, and the calibration intensity value is 0 HU and is an intensity value for which a calibration of the scanner apparatus, in this case the CT scanner, has previously been performed. In other embodiments the calibration intensity value may be any intensity value for which the scanner performs intensity measurements that are known, or likely, to be accurate. Each sub-region does not have to consist of a single voxel, or pixel, and instead for example each sub-region under consideration may comprise a plurality of voxels, or pixels, if desired.

It is a feature of certain embodiments that the voxels or pixels within a bone region and identified as having an intensity value close to (e.g. within a threshold intensity range of) 0 HU can be used to determine whether the bone region has a medical condition, for example is of low quality.

Regions of bones which comprise low quality bone structure usually have larger cavities than healthy bones resulting in a lower density and a consequent lower opacity to radiation. The reduction in solid bone tissue can be detected using an imaging technology that is sensitive to the density of bone or the proportion of solid matter to non-solid matter inside a bone.

X-rays are particularly sensitive to dense structures like bone. A CT scan comprises a series of X-ray images and is a good candidate for assessing bone quality due to the high attenuation experienced by X-rays in bones. Images from a CT scan may comprise attenuation values as a function of position, or may be rendered to comprise a parameter derived from attenuation as a function of position. The Hounsfield (HU) scale is a scale for describing radiodensity of a material which is related to the attenuation presented by the material. The radiodensity of distilled water at standard temperature and pressure is usually defined as zero Hounsfield units (HU) while the radiodensity of air at standard temperature and pressure is defined as-1000 HU.

CT scanners are usually calibrated for both air and distilled water at standard temperature and pressure. Standard values of pressure and temperature for CT scanner calibration may be defined as twenty degrees Celsius and one atmosphere of pressure. An attenuation measurement at either 0HU or −1000 HU is a robust empirical result as it is not dependent on the transmission spectrum of the X-ray beam source or the characteristics of the detector. An attenuation measurement at 0 HU is well below the attenuation of normal healthy bone. An attenuation measurement at 0 HU is particularly relevant as bone pathology, such as osteoporosis and osteopenia, results in low density areas in bone tissue which present attenuation values close to and/or below 0 HU. This may be due to the trabecular structure of bone breaking down or demineralising.

The voxels or pixels within a bone region identified at stage 46 can be used to determine whether bone is of low quality or otherwise suffers from a medical condition.

Any suitable threshold range of values may be used to identify relevant pixels or voxels, or other sub-regions, that in turn are used to identify bone regions that are of low bone quality.

Working at a low value of attenuation avoids the variability of measured attenuation values between different scanner and/or scanners with different kilovoltage peak (kVp) values when imaging material with high attenuation. Less well calibrated ranged of the CT scanner can be avoided by measuring the absence of bone material, or the presence of holes, rather than attempting to quantify the density of the interrogated bone material. In stage 46, the medical images comprising the medical imaging dataset may be analysed as a whole or only the segmented bone tissue may be analysed.

The output of stage 46 is a dataset comprising the positions of sub-regions (e. g voxels or pixels) in the segmented image data, wherein the intensity and/or attenuation of a region/pixel/voxel is below one or more reference intensities and/or attenuations that correspond to one or more reference intensity and/or attenuation values. The reference attenuation and/or intensity values may comprise calibration attenuation and/or calibration intensity values associated with the equipment used for medical imaging.

Stage 48 comprises computing the number and/or proportion and/or concentration of low quality bone tissue in individual bones, for example, in each vertebra. This can be done by defining a total area or a volume in images or in a region of the images comprising bone tissue that comprises low intensity and/or low attenuation (low HU) regions.

In some embodiments, low HU pixels or voxels may be counted for each vertebra for the case of two-dimensional or three-dimensional medical images respectively. The two-dimensional image data may comprise a two-dimensional slice from a tomographically acquired three-dimensional CT image. The area/volume or the number of pixels/voxels may be represented as a ratio or percentage of the total area/volume or number of pixels/voxels comprising each bone. The concentration of sub-regions comprising low quality bone tissue may be determined for all the bone regions in the imaging data or for individual bones, such as for individual vertebra. Concentration of sub-regions may be determined for the full spinal column or a subset of the full spinal column, such as the cervical spine, the thoracic spine and the lumbar spine. The concentration of sub-regions within one or more bone regions may be displayed on the output device 16 for the benefit or a user/operator.

At stage 50, a dataset is generated comprising the spatial distribution of sub-regions comprising low quality bone tissue in the medical image data. This dataset may be represented in the form of one or more medical images overlaid with visual indicators of segmentation of the medical image with respect to bone tissue and further overlaid with visual indications of the sub-regions where bone quality is low, as assessed by the intensity and/or attenuation presented by the sub-regions. This dataset may be stored for further processing and analysis and may also be displayed for the benefit of a user/operator on an output device 16.

At stage 50, results of stage 48 are compared with expected results to determine the bone health of individual bones, for example, individual vertebrae. The processing apparatus may be used to determine the presence or absence of a medical condition, such as osteoporosis, osteopenia, vertebral compression fractures and the risk of future fractures/re-fractures.

The determination of one or more medical conditions may depend on the concentration of sub-regions within one or more bone regions and/or the distribution of sub-regions within one or more bone regions. The processing apparatus 14 may be provided with data that provides typical values and/or ranges of the number or proportion of low attenuation regions in individual bones that signify low bone quality, healthy bone quality and other possible classifications of bone quality.

The processing apparatus 14 may be provided with threshold concentration values of sub-regions for one or more bone regions. The threshold concentration values may be different for different bone regions, such as for different vertebral bones. The threshold concentration values may vary based on at least one of the sex, weight, body mass index and ethnicity of the subject of the medical imaging data. The threshold concentration values may be compared to the concentration of sub-regions determined in stage 48. The threshold concentration values may be in the form of a ratio, for example a ratio of low attenuation sub-regions to total area/volume. The threshold concentration value may be a single number expressing the prevalence of low quality bone tissue as a number of pixels, voxels or units of area and volume as applicable.

In some embodiments, the quality of bone tissue may be graded into more than two classifications. There may be more classifications of bone quality which correspond to further threshold values of attenuation on the Hounsfield scale. For example, regions in the image data that are associated with an attenuation below 0HU may represent bad quality bone tissue whereas regions that are associated with an attenuation between 10HU and 0HU may be considered to be of intermediate quality. Regions associated with an attenuation above 10HU may be considered healthy bone tissue. In other examples, there may be more or less classifications at the same or other threshold attenuation points. Stage 52 comprises providing an output to the user. The output of stage 52 may comprise a list of the labels of bones and indications of whether the quality of bone tissue in the labelled bones is within one or more threshold attenuation ranges or values from a reference attenuation. The output of stage 52 may comprise any combination of the parameters/distribution determined in stages 46 and 50.

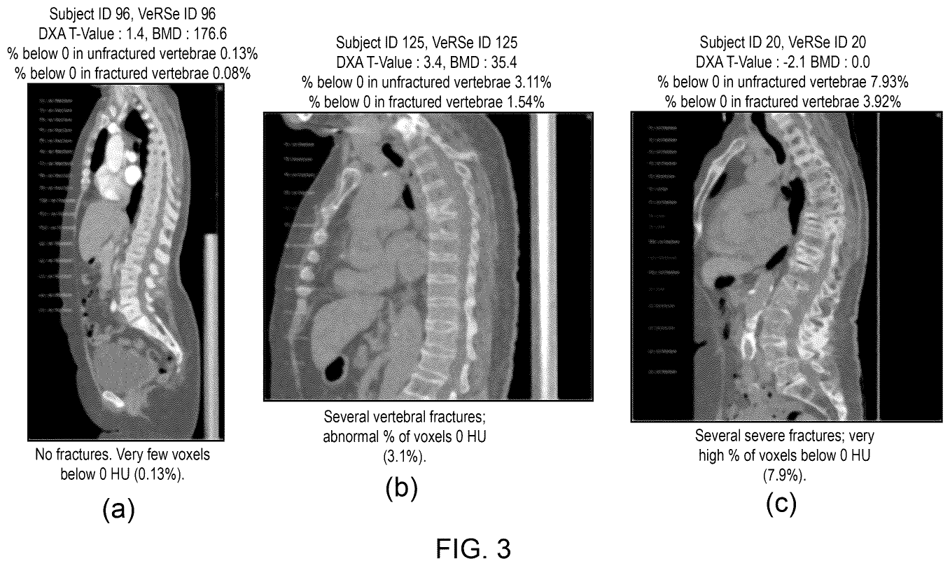

FIG. 3 (a), 3 (b) and 3 (c) shows three medical images obtained using X-ray imaging showing all or part of a subject's spinal column. The ground truth for the prevalence of fractures in vertebrae of each subject's spinal column is known and expressed in text accompanying the label of each vertebra on the left hand side of FIG. 3 (a), 3 (b) and 3 (c). The images are segmented based on ground truth to ensure that imperfect segmentation does not lead to false density effects.

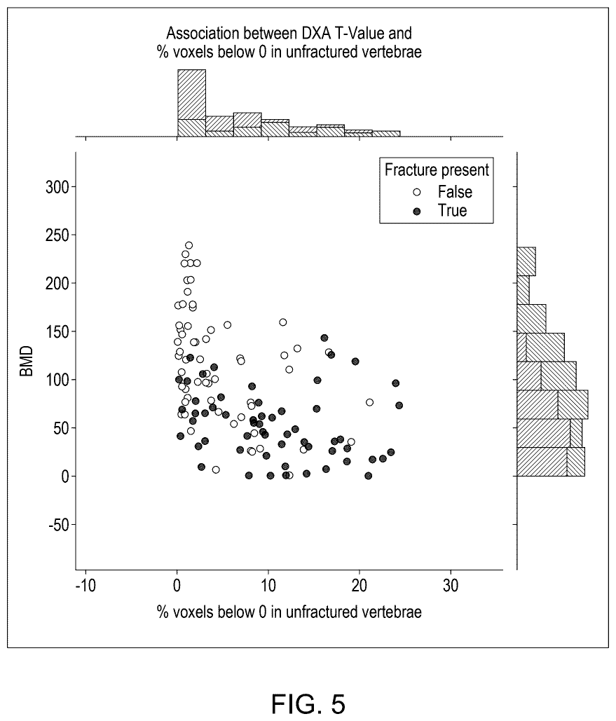

Segmented bone regions are highlighted as are sub-regions comprising pixels that present an attenuation at or below 0 HU. The subject's bone mineral density (BMD) and T-score have previously been measured, using a DEXA imaging device. Progressing from FIG. 3 (a) to 3 (c), bone quality of the subjects generally decreases. While the subject in FIG. 5 (a) has no vertebral fractures, the subjects of FIG. 3 (b) and 3 (c) have an increasing number and severity of vertebral fractures. The images are processed using method 40 and overlaid with indications of regions/pixels that exhibit an intensity which corresponds to attenuation of 0 Hounsfield units or lower. FIG. 3 (a) shows a subject with no vertebral fractures, a BMD of 176.6, a T-Score of 1.4 and with 0.13% of image pixels in unfractured vertebrae below 0 HU. FIG. 3 (b) shows a subject with several vertebral fractures, a BMD of 35.4, a T-Score of −3.4 and with 3.1% of image pixels in unfractured vertebrae below 0 HU. FIG. 3 (c) shows a subject with several severe vertebral fractures, a BMD of 0, a T-Score of −2.1 and with 7.9% of image pixels in unfractured vertebrae below 0 HU. The subjects in FIG. 3 (a), 3 (b) and 3 (c) have 0.08%, 1.5% and 3.9% pixels presenting attenuation below 0 HU in fractured vertebrae respectively. It can be seen that subjects with low BMD and that have vertebral fractures in the other vertebrae have a higher concentration of pixels below 0 HU in both fractured and unfractured vertebrae, especially in the lumbar region. Fractured vertebrae often show higher density rather than lower, due to the compression of the remaining bone.

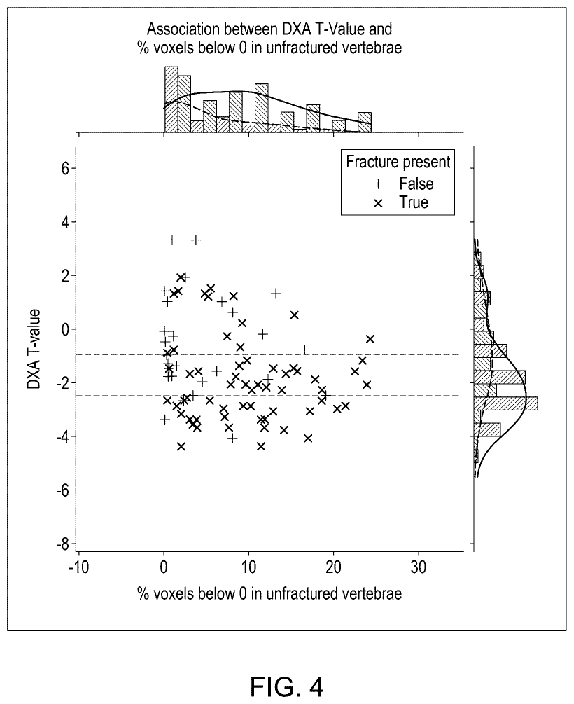

FIG. 4 is a plot of T-value versus percentage of voxels below 0 HU and represents the results of a study conducted to understand the relationship between the two parameters. Bone density can be measured as a T-score. A DEXA scan is used to obtain bone density which is compared to the ‘average’ or normal range of bone density found in a healthy young woman. A T-score above −1 means that bone density is in a normal range. A T-score between −1 and −2.5 means that bone density is lower than average and the subject may suffer from Osteopenia but it is not low enough to meet the criteria for osteoporosis. A T-score below −2.5 means that the bone density is in a range that is low enough that the subject may be suffering from osteoporosis. In FIG. 4, T-score or T-value measured using DEXA is plotted against the percentage of voxels in vertebrae that exhibit attenuation lower than 0 HU in the results of a CT scan. The horizontal dashed lines represent T-values of −1 and −2.5 demarcating the regions that signify normal bone density, osteopenia and osteoporosis. Fractured vertebrae are not considered in this plot. This is because some fractures can result in a higher than normal bone density, for example, due to compression. Vertebrae that do not have fractures are represented with crosses on the plot. Vertebrae that do have fractures are also represented on the plot, with different crosses.

It can be seen that a number of data points for vertebrae with no fracture appear above the horizontal lines that represents a T-value of −1. These are from subjects with no fractures, high T-values but that present attenuation below 0HU. These could be due for example to:
(a) a subject who will experience a vertebral fracture imminently
(b) poor quality or noisy images
(c) spine segmentation failures

In practice, noisy images may be filtered automatically. Spine segmentation failures are unlikely to be the cause of the false positive detections in FIG. 4 since the segmentation was verified against ground truth for the study. It can be seen that there are many subjects with good DEXA T-scores greater than zero who have had fractures.

FIG. 5 is a plot of BMD (in grams per square centimetre) versus percentage of voxels in unfractured vertebrae for subjects that have an attenuation below 0 HU and represents the results of a study conducted to understand the relationship between the two parameters. A DEXA scan is used to obtain BMD. In FIG. 5, BMD is plotted against the percentage of voxels in vertebrae that exhibit attenuation lower than 0 HU in the results of a CT scan. Vertebrae that do not have fractures are represented by dots on the plot. Vertebrae that have fractures are represented by different dots on the plot. It can be seen that most fractures occur for low values of BMD. Furthermore, Most fractures have larger percentages of low density voxels. There are several examples of high BMD wherein fractures exist and many of these show high percentages of low density voxels.

In some embodiments, the processing circuitry may be configured to reduce noise in the imaging dataset, for example before providing the resulting noise-reduced data set to stage 42 for processing in the method of FIG. 2. The circuitry may use low-pass filtering, anisotropic smoothing and other such techniques to reduce the noise in images. Images with noise above a predetermined threshold may be identified by inspecting imaging parameters related to the measuring equipment. In the case of CT scans, these imaging parameters may comprise parameters of the X-ray dose used during scanning, such as the X-ray tube voltage and current. It may be the case that high levels of X-ray radiation results in noisy images. The amount of noise in an image may be determined by analysing noise in homogeneous regions of an image, such as regions comprising the liver or aorta. The homogeneity of tissue in such area may imply that any sub-regions detected in these or similar areas indicate high levels of noise in the imaging data or in the particular image that comprises these regions. The detection of a noisy image may be followed by processing the image to reduce or compensate for the noise in the image or the exclusion of the image from processing.

In other embodiments, the processing circuitry may be configured to detect common artefacts in the imaging data. The presence of artefacts may be determined based on their density or radiodensity as measured by the imaging apparatus. These may comprise extraneous objects such as metal implants, pacemakers, wire leads and other such objects. These artefacts may be restricted to a range of image slices containing the high-density objects and may, for instance, be detected using intensity-based statistics from all vertebrae and detecting outliers. Affected vertebrae may be excluded from further analysis.

In some embodiments, vertebral shape metrics may be provided to the processing circuitry to detect vertebral compression fractures (VCFs). Vertebrae with VCF(s) may have anomalously fewer low-density voxels or sub-regions due to the compression of the trabecular bone structure resulting in an increase in density. The spatial distribution of sub-regions within bone regions or individual vertebrae may be used to detect bone structure with a higher risk of future fracture, for instance using methods such as the grey-level co-occurrence matrix, grey-level run-length analysis or similar.

The medical conditions as mentioned herein, or any other suitable medical conditions associated with bone tissue health, may be determined as an incidental finding in a CT imaging workflow. Chest X-rays and abdomen X-rays may incidentally include images of bone regions, such as some or all of a spinal column. Such X-ray images may be analysed using the methods and apparatus disclosed herein to detect bone health quality from scans where bone health was not the pathology under investigation. The methods and apparatus described may form an additional input to a fracture/re-fracture risk calculation (c.f. FRAX score), especially where a DEXA measurement is not available. They may be used to measure significant longitudinal changes in bone structure, for instance, in patients undergoing treatment for cancer who have regular CT scans and treatments that impact bone strength or post VCF corrective surgery.

Certain embodiments provide methods that exploit the calibration points of CT scanners by identifying regions of low attenuation and consequently low density in bones in a range of attenuation values around 0 HU where results across scanners are more consistent rather than attempting to quantify high HU values which are more scanner/energy dependent. A resulting distribution of the low density voxels in the CT images contains structural information related to bone strength which may be provided to the user and/or stored in a memory device. The spatial distribution of low density voxels provides more robust data than mean HU values for individual bones, such as individual vertebra. Since limited contrast material is absorbed in bones, such methods are suitable for either non-contrast or contrast-enhanced CT scan images. The method can be applied to CT images including photon counting CT images. It is easy to automate and can evaluate secondary bone structure in scans that are targeted towards a different primary bone structure. It may also be used as a dedicated method for scanning and analysing bone quality, for example, in the spinal column.

According to various embodiments there is provided a medical image processing apparatus comprising processing circuitry configured to:
receive a medical imaging data set comprising intensity values as a function of position;
obtain a segmentation of a bone region represented in the medical imaging data set;
process the medical imaging data set, for example to identify a plurality of sub-regions within the bone region for which intensity values are within a threshold intensity range about a calibration intensity value; and
   determine concentration and/or distribution of the sub-regions within the bone region.

The processing circuitry may further determine the presence or absence of a medical condition in dependence on the concentration and/or distribution of the identified sub-regions within the bone region. The medical condition may be osteopenia or osteoporosis.

The processing circuitry may output a representation of the concentration and/or distribution of the identified sub-regions within the bone region for the benefit of the user or may store this data to memory. The bone region represented in the medical imaging dataset may comprise one or more vertebra.

The segmentation received by or determined by the apparatus may include one or more labels identifying the bone region. These may be labels associated with individual vertebra. Each identified sub-region may be a pixel or voxel.

The processing apparatus may compare the determined concentration of sub-regions with one or more threshold concentration values. The threshold concentration values may be associated with individual vertebra. The threshold concentration values may be based on the characteristics of the bone region, and may hence differ for each vertebra. The threshold concentration values may depend on one or more characteristics of the subject of the medical imaging dataset, such as the age, sex, weight, height, body mass index, ethnicity and other such characteristics of the subject.

The calibration intensity value may comprise zero Hounsfield units and/or a calibration value representing water. Water may be at a standard temperature and pressure for CT scanner calibration, wherein standard temperature and pressure for CT scanner calibration is twenty degrees Celsius and one atmosphere of pressure respectively, or 293.15 Kelvin and 1.013×10⁵ Pascals. Sub-regions may be identified within bone regions based on their intensity being lower than zero Hounsfield units and/or a calibration value representing water. Sub-regions may also be identified based on other calibration values or other attenuation values.

The medical imaging dataset may be at least partially obtained from a computer tomography (CT) scan. This may include a micro-CT scan. The processing circuitry may be configured to reduce noise in the medical imaging dataset and may use one or more of low-pass filtering, anisotropic smoothing or other such methods are used to reduce noise before processing. Images with noise beyond a threshold may be excluded from processing. The processing circuitry may be configured to detect images comprising extraneous structures and exclude them from processing. This may include high density objects like metal implants, pacemakers, wire leads and similar objects.

The processing circuitry may further determine the presence of fractures in dependence on the concentration and/or distribution of the identified sub-regions within the bone region. The processing circuitry may be configured to analyse the distribution of the sub-regions within the bone region in order to assess the risk of future fracture/re-fracture of the bone region.

The processing circuitry may be configured to determine the concentration and/or distribution of the sub-regions within the bone region using medical imaging data obtained during imaging wherein the purpose of the imaging is other than to detect a medical condition of the bone region. In this way, an assessment of bone health or fracture risk may be an incidental finding in a scan meant to detect a related or unrelated pathology.

The processing circuitry may be configured to detect changes in the dimensions of the bone region on the basis of medical imaging data obtained from a subject at two or more different points in time. In this way, the circuitry may be able to measure significant longitudinal changes in bone structure, such as due to vertical compression fractures. This feature may also be used for patients undergoing treatment for cancer who have regular CT scans and whose treatment impacts bone strength. It may also be used to monitor impact of surgery to correct vertical compression fractures.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. A medical imaging apparatus, comprising:
processing circuitry configured to:
receive a medical imaging data set comprising intensity values as a function of position;
obtain a segmentation of a bone region represented in the medical imaging data set;
process the medical imaging data set to identify a plurality of sub-regions within the bone region for which intensity values are within a threshold intensity range with respect to a calibration intensity value; and
determine a concentration and/or a distribution of the sub-regions within the bone region.

2. The apparatus of claim 1, wherein the processing circuitry is further configured to determine the presence of a medical condition in dependence on the concentration and/or distribution of the identified sub-regions within the bone region.

3. The apparatus of claim 2, wherein the medical condition comprises osteopenia and/or osteoporosis.

4. The apparatus of claim 1, wherein the processing circuitry is further configured to output a representation of the concentration and/or distribution of the identified sub-regions within the bone region.

5. The apparatus of claim 1, wherein the bone region comprises one or more vertebrae.

6. The apparatus of claim 1, wherein the segmentation includes one or more labels identifying the bone region.

7. The apparatus of claim 1, wherein each sub-region is a pixel or voxel.

8. The apparatus of claim 1, wherein the processing apparatus compares the determined concentration with one or more threshold concentration values.

9. The apparatus of claim 8, wherein the threshold concentration values are based on the characteristics of the bone region and/or at least one characteristic of the subject of the medical imaging dataset.

10. The apparatus of claim 9, wherein the characteristic of the subject includes one or more of the age, sex, weight, height, body mass index, ethnicity, or other characteristic.

11. The apparatus of claim 1, wherein the calibration intensity value comprises zero Hounsfield units and/or a calibration value representing water.

12. The apparatus of claim 1, wherein sub-regions are identified within bone regions based on their intensity being lower than zero Hounsfield units and/or a calibration value representing water.

13. The apparatus of claim 1, wherein the medical imaging dataset is a CT data set.

14. The apparatus of claim 1, wherein the processing circuitry is further configured to reduce noise in the medical imaging dataset.

15. The apparatus of claim 14, wherein one or more of low-pass filtering or anisotropic smoothing are used to reduce the noise.

16. The apparatus of claim 1, wherein the processing circuitry is further configured to detect images comprising extraneous structures and exclude the images from processing.

17. The apparatus of claim 1, wherein the processing circuitry is further configured to determine a presence of fractures in dependence on the concentration and/or distribution of the identified sub-regions within the bone region.

18. The apparatus of claim 1, wherein the processing circuitry is further configured to analyze the distribution of the sub-regions within the bone region in order to assess the risk of future fracture/re-fracture of the bone region.

19. The apparatus of claim 1, wherein the processing circuitry is further configured to detect changes in the dimensions of the bone region on the basis of medical imaging data obtained from a subject at two or more different points in time.

20. A medical imaging processing method, comprising:
receiving a medical imaging data set comprising intensity values as a function of position;
obtaining a segmentation of a bone region represented in the medical imaging data set;
processing the medical imaging data set to identify a plurality of sub-regions within the bone region for which intensity values are within a threshold intensity range with respect to a calibration intensity value; and
determining concentration and/or distribution of the sub-regions within the bone region.
